(19) United States
(12) Patent Application Publication
(10) Pub. No.: US 2017/0340265 A1
(43) Pub. Date: Nov. 30, 2017

(54) WIRELESS SMART ADULT TOY SYSTEM

(71) Applicant: WEN-HUNG SHEN, TAIPEI (TW)

(72) Inventor: WEN-HUNG SHEN, TAIPEI (TW)

(21) Appl. No.: 15/162,762

(22) Filed: May 24, 2016

(51) Int. Cl. A61B 5/00; H04M 1/725; A61H 19/00; H04W 4/00

(52) U.S. Cl. CPC A61B 5/43; H04W 4/008; H04M 1/72525; A61H 19/00; A61H 19/34; A61H 19/32; A61H 19/44

(57) ABSTRACT

A wireless smart adult toy system includes a smart health monitoring device to be worn by a user to detect and collect physical and physiological data of the body of the user continuously at a predetermined time interval, a smart control device mounted in the smart health monitoring device for receiving, analyzing and computing the physical and physiological data and then producing a corresponding control signal, and an adult toy mountable around the sexual organ of the user and drivable by the control signal in a wireless manner to make different movements at different degrees of intensity and rhythm and/or to produce different sound and light in response to the behaviors of the user.

### FIELD OF INVENTION

The present invention relates to adult toy technology and more particularly, to a wireless smart adult toy system, which uses a smart health monitoring device to detect and collect physical and physiological data of the body of the user continuously at a predetermined time interval and then to provide the data to a smart control device for analysis and calculation so further provide a wireless control signal to an adult toy that is mounted around the sexual organ of the user, causing the adult toy to move at different degrees of intensity and rhythm and/or to produce different sound and light, and thus, achieving enhanced sexual interest and increasing the probability of orgasm.

### BACKGROUND

Many sex toys and aids for personal use or for use between sexual partners are known. However, these sex toys and aids cannot detect the physical and physiological conditions of the body of the user and then analyze and compute the detected data for making changes in operation to achieve enhanced sexual interest and to increase the probability of orgasm. In general, conventional sex toys and aids cannot help a pair of sex couple to achieve sexual matching behaviors in a natural and accurate manner and to increase the incidence of orgasm.

### SUMMARY OF THE DESCRIPTION

The present invention has been accomplished under the circumstances in view. It is main object of the present invention to provide a wireless smart adult toy system, which comprises a smart health monitoring device to be worn by a user to detect and collect physical and physiological data of the body of the user continuously at a predetermined time interval, a health monitoring device mounted in the smart health monitoring device and adapted for receiving the physical and physiological data from the smart health monitoring device, analyzing and computing the physical and physiological data and then producing a corresponding control signal, and an adult toy mountable around the sexual organ of the user and drivable by the control signal in a wireless manner to produce different movements at different degrees of intensity and rhythm and/or to produce different sound and light in response to the behaviors of the user.

It is another object of the present invention to provide a wireless smart adult toy system, which comprises a smart health monitoring device to be worn by a user to detect and collect physical and physiological data of the body of the user continuously at a predetermined time interval, and a smart control device mounted in the smart health monitoring device and adapted for receiving the physical and physiological data from the smart health monitoring device, analyzing and computing the physical and physiological data and then producing sound and light in response to changes of the state of the body of the user.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is a system block diagram of a wireless smart adult toy system in accordance with the present invention.

FIG. 2 is a schematic drawing illustrating the wireless smart adult toy system used in a female user in accordance with the present invention.

FIG. 3 illustrates the hardware devices of the wireless smart adult toy system shown in FIG. 2.

FIG. 4 is a schematic drawing illustrating the wireless smart adult o system used in a male user in accordance with the present invention.

FIG. 5 illustrates the hardware devices of the wireless smart adult toy system shown in FIG. 4.

FIG. 6 is a system block diagram of an alternate form of the wireless smart adult toy system in accordance with the present invention.

### DETAILED DESCRIPTION

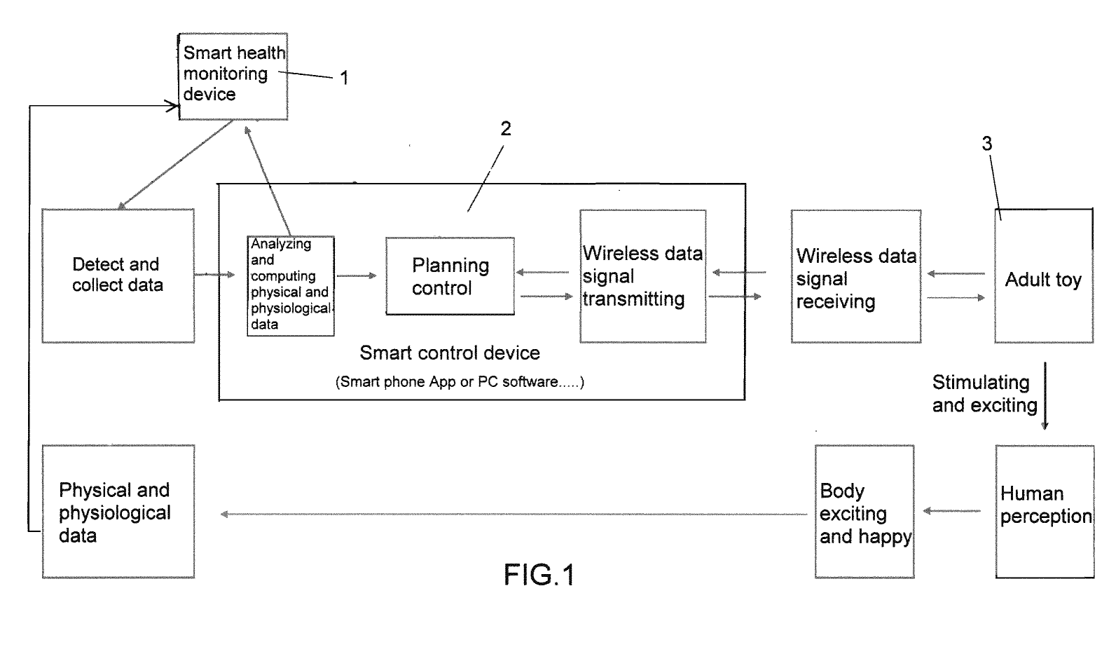

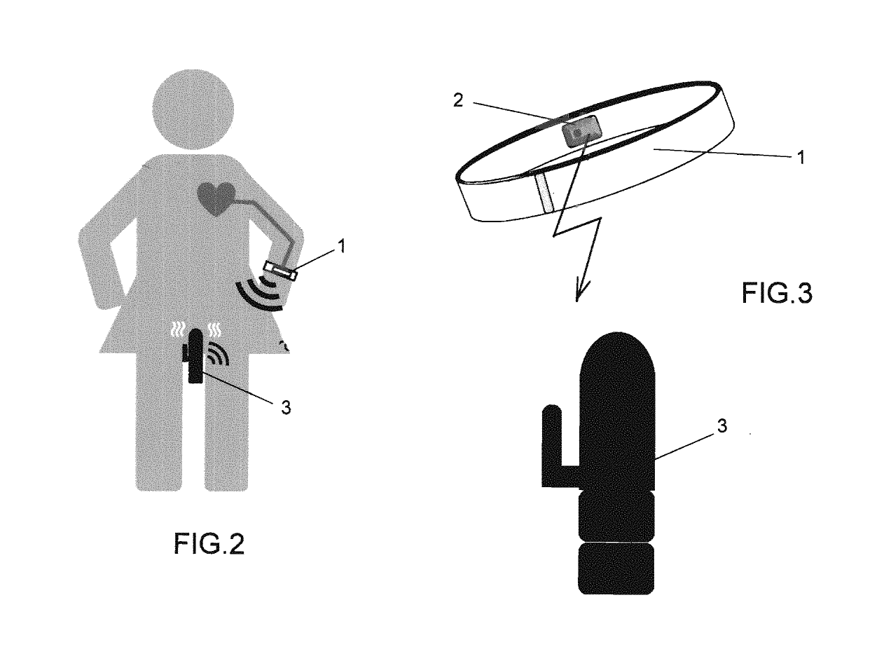

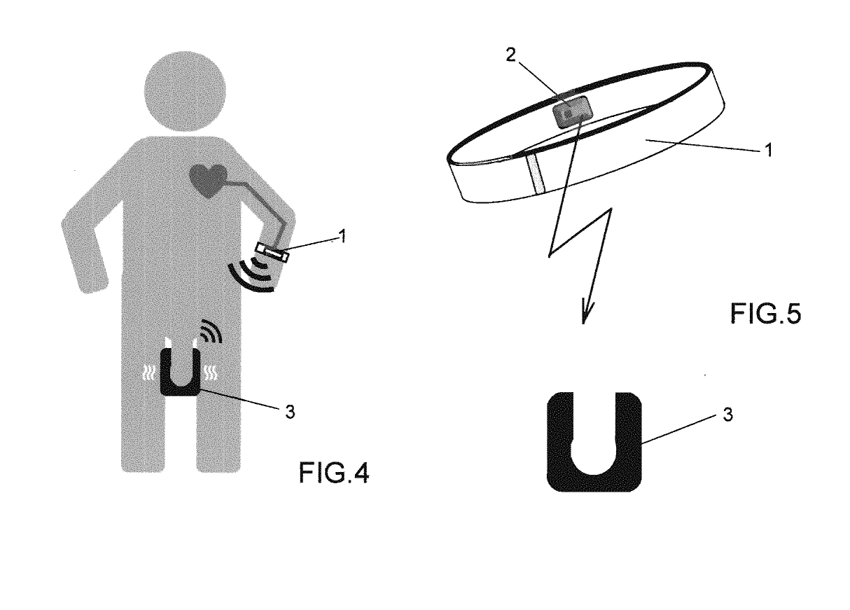

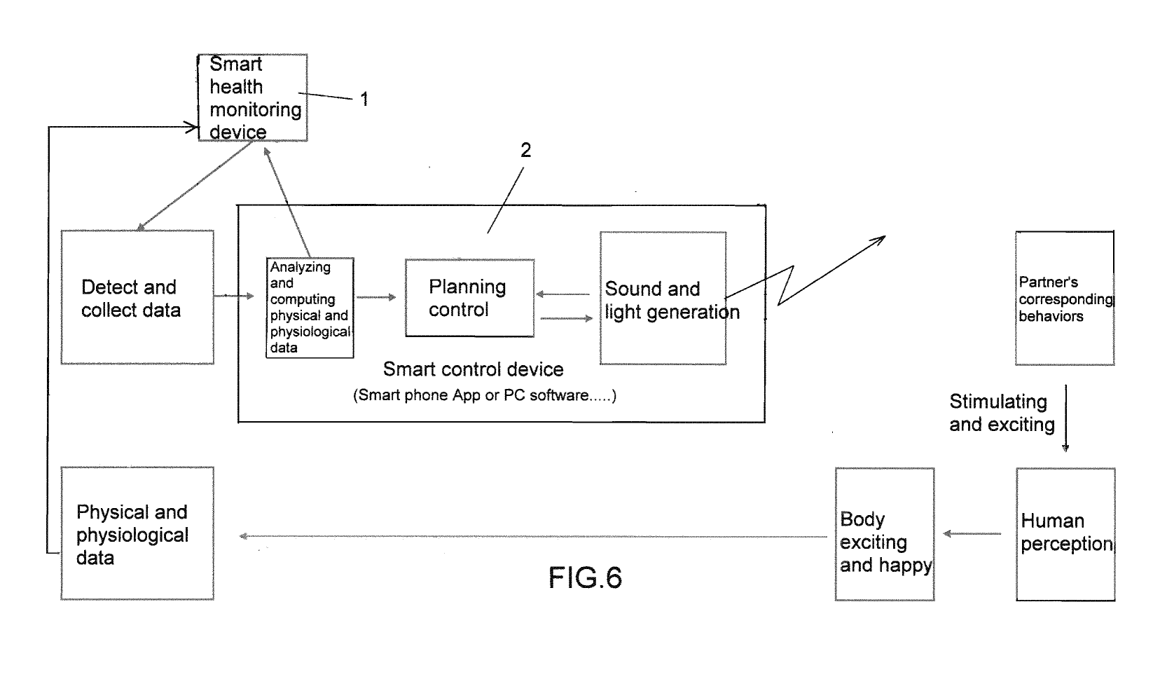

Referring to FIGS. 1-6, a wireless smart adult toy system in accordance with the present invention is shown. The wireless smart adult toy system comprises a smart health monitoring device (1), a smart control device (2) and an adult toy (3). The smart health monitoring device (1) is made in the form of, for example, wrist watch for wearing by a user and adapted for detecting and collecting physical and physiological data of the body of the user. The smart control device (2) is a wireless intelligent data processing device mounted in the smart health monitoring device (1), having built therein a smart phone application software or computer software. The smart control device (2) has built therein an application software that allows for updates. The smart control device (2) is adapted for reading, analyzing and computing the data provided by the smart health monitoring device (1), so as to further implement a related planning control, driving the adult toy (3) in a wireless manner to make different movements at different degrees of intensity and rhythm and/or to produce different sound and light in response to the behaviors of the partner.

When a user wears and turns on the smart health monitoring device (1), the smart health monitoring device (1) continuously detects and collects physical and physiological data of the body of the user at a predetermined time interval, and transmits collated data to the smart control device (2) in a wireless manner or directly, enabling the smart control device (2) to analyze and compute the data and to further produce a control signal for driving the adult toy (3) in a wireless manner to make different movements at different degrees of intensity and rhythm and/or to produce different sound and light in response to the behaviors of the partner, thereby exciting the user and making the user feel happy physically as well as mentally. During application, the smart health monitoring device (1) repeatedly detects and collets physical and physiological data of the body of the user and transmits each collated data to the smart control device (2) for analysis and further planning control implementation, achieving enhanced sexual interest and sexual matching behaviors in a natural and accurate manner and increasing the incidence of orgasm.

When a female user wears the smart health monitoring device (1) and turns it on, the smart health monitoring device (1) continuously detects and collects physical and physiological data of the body of the female user at a predetermined time interval, and transmits collated data to the smart control device (2) in a wireless manner or directly, enabling the smart control device (2) to analyze and compute the data and to further produce a control signal for driving the adult toy (3) in a wireless manner to make different movements at different degrees of intensity and rhythm and/or to produce different sound and light, giving signal to her sex partner to make a corresponding movement so as to stimulate excitement. At this time, any physical change will be continuously detected by the smart health monitoring device (1) and provided to the smart control device (2) for analysis and further planning control implementation, achieving enhanced sexual interest and sexual matching behaviors in a natural and accurate manner and increasing the incidence of orgasm.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A wireless smart adult toy system, comprising:
a smart health monitoring device to be worn by a user to detect and collect physical and physiological data of the body of said user continuously at a predetermined time interval;
a smart control device mounted in said smart health monitoring device and adapted for receiving said physical and physiological data from said smart health monitoring device, analyzing and computing said physical and physiological data and then producing a control signal; and
an adult toy mountable around the sexual organ of said user and drivable by said control signal in a wireless manner to make different movements at different degrees of intensity and rhythm and/or to produce different sound and light in response to the behaviors of said user.

2. The wireless smart adult toy system as claimed in claim I, wherein said smart control device is linkable to said smart health monitoring device in a wireless manner.

3. The wireless smart adult toy system as claimed in claim 1, wherein said smart control device has built therein an application software that allows for updates.

4. A wireless smart adult toy system, comprising:
a smart health monitoring device to be worn by a user to detect and collect physical and physiological data of the body of said user continuously at a predetermined time interval; and
a smart control device mounted in said smart health monitoring device and adapted for receiving said physical and physiological data from said smart health monitoring device, analyzing and computing said physical and physiological data and producing sound and light in response to changes of the state of the body of said user.

5. The wireless smart adult toy system as claimed in claim 4, wherein said smart control device is linkable to said smart health monitoring device in a wireless manner.

6. The wireless smart adult toy system as claimed in claim 4, wherein said smart control device has built therein an application software that allows for updates.
